(19) United States
(12) Patent Application Publication
(10) Pub. No.: US 2018/0334548 A1
(43) Pub. Date: Nov. 22, 2018

(54) ANTIMICROBIAL FLUOROPOLYMER

(71) Applicants: DAIKIN AMERICA, INC., Orangeburg, NY (US); DAIKIN INDUSTRIES, LTD., Osaka (JP)

(72) Inventors: Qibo JIANG, Decatur, AL (US); Arthur MARTIN, Decatur, AL (US); Keith SHIH, Decatur, AL (US)

(73) Assignees: DAIKIN AMERICA, INC., Orangeburg, NY (US); DAIKIN INDUSTRIES, LTD., Osaka (JP)

(21) Appl. No.: 15/983,554

(22) Filed: May 18, 2018

Related U.S. Application Data
(60) Provisional application No. 62/508,544, filed on May 19, 2017.

(51) Int. Cl. C08K 3/08; C08J 3/20; A61L 29/04; A61L 29/16; A01N 59/16; A01N 25/10

(52) U.S. Cl. CPC C08K 3/08; C08J 3/203; A61L 29/041; A61L 29/16; A01N 59/16; A01N 25/10; C08K 2201/019; A61L 2300/404; C08J 2323/08; C08J 2327/18; C08J 2323/28; C08J 2327/20; C08K 2003/0806

(57) ABSTRACT

Provided is a composition having excellent antimicrobial performance. The present invention relates to a composite material, comprising: a fluoropolymer; and an antimicrobial agent; wherein the fluoropolymer and the antimicrobial agent are compounded.

### BACKGROUND OF THE INVENTION

A fluororesin has excellent characteristics such as thermal resistance, chemical resistance, solvent resistance, and insulation properties. For this reason, a fluororesin is molded into various products such as tubes, pipes, thin films, and filaments by, for example, melt extrusion molding, and such products have been made commercially available.

In particular, fluoropolymers such as fluorinated ethylene propylene copolymer (or equivalently, a copolymer of tetrafluoroethylene and hexafluoropropylene; “FEP”) are used in the medical, industrial, and food industries, for example, in the form of a tube/hose or catheter for transferring liquids and in the form of packaging film, pouch, and bottles. In these cases, microbes such as mold, mildew, bacteria, and fungi can contaminate these articles when in use, which can hamper the applications of these melt-processible flurorpolymers. Development of fluoropolymers having antimicrobial capabilities will avoid this problem.

Antimicrobial fluoropolymer materials treated with chitosan are disclosed in U.S. Pat. No. 8,043,632 B2, which is assigned on its face to E.I. duPont de Nemours and Company, and the content of which is hereby incorporated by reference. The fluoropolymers in this patent already contain amino-reactive functional groups as polymerized (for example, a fluoropolymer copolymerized with a maleic anhydride graft monomer, or a perfluorinated ionomer with metal ions) that can react with the chitosan amine groups. However, fluoropolymers copolymerized with a graft monomer that can react directly with chitosan amino groups are not commercially available.

In U.S. Pat. No. 7,217,754 B2, which is assigned on its face to Integument Technologies, and the content of which is hereby incorporated by reference, a composite of an inorganic macromolecular network having the formula (—Mo—Mo—)ₙ, where n is from about 10,000 to about 10⁶ and Mo is a Group IIIa, IVa, Va, VIa, VIIa, or VIIIa metal, was compounded with fluoropolymer to attain an antimicrobial capability.

U.S. Pat. No. 8,685,424 B2, which is assigned on its face to Zeus Industrial Products, and the content of which is hereby incorporated by reference, disclosed a method for making a nonwoven mat containing one or more antimicrobial agents. More specifically, it contains a fluorinated polymer, a fiberizing polymer, one or more antimicrobial agents, and a solvent. Silver nanoparticles are specially mentioned as the antimicrobial agents.

### BRIEF SUMMARY OF THE INVENTION

Fluoropolymers themselves have no capability of killing microorganisms or inhibiting their growth. Therefore, under certain conditions, articles made by fluoropolymers can be contaminated with microbes, which is not desirable for their commonly intended uses. However, by introducing antimicrobial agents, such as zeolite supporting a metal, into the base fluoropolymer resins, the resulting product and the articles made from it will gain the capability of killing bacteria or slowing down or stalling bacterial growth. The zeolite supporting a metal contains elemental ions of silver, copper, zinc, or a combination of these elements as active antimicrobial ingredients in zeolite carriers. In the present invention, fluoropolymers and antimicrobial agents may be mixed and compounded in a weight ratio of between 90:10 and 99.99:0.01, more preferably between 95:5 and 99.9:0.1, and most preferably between 97:3 and 99.8:0.2.

The metal in the zeolite supporting a metal may be a metal having antimicrobial performance. For example, the metal may be at least one selected from the group consisting of copper, zinc, and silver, and is preferably silver.

The metal may be supported in the form of metal ions.

The proportion of the metal (metal ions) supported by the zeolite is preferably 1 to 30 mass %, mote preferably 25 mass % or less, while more preferably 4 mass % or more, relative to the zeolite supporting a metal.

Examples a fluoropolymers for the purpose of the present invention include, but are not limited to, FEP, a copolymer of ethylene and tetrafluoroethylene (“ETFE”), a perfluoroalkoxy polymer (“PFA”), polyvinylidene fluoride (“PVDF”), a terpolymer of ethylene, tetrafluoroethylene, and hexafluoropropylene (“EFEP”), other terpolymers of these monomers, polytetrafluoroethylene (“PTFE”) in the form of dispersions (typically, but not necessarily, aqueous dispersions of stabilized minute PTFE particles), PTFE in the form of fine powders (typically, but not necessarily, produced from emulsion polymerization), and PTFE in the form of molding powders (typically, but not necessarily, characterized by high melting point, low friction, and chemical inertness).

For the purpose of the present invention, the monomer molar ratio of tetrafluoroethylene to hexafluoropropylene of the FEP may be between 70:30 and 99:1, and more preferably between 80:20 and 97:3. The monomer molar ratio of ethylene to tetrafluoroethylene of the ETFE may be between 80:20 and 10:90, more preferably between 63:37 and 15:85, and most preferably between 62:38 and 20:80. The monomer molar ratio of ethylene, tetrafluoroethylene, and hexafluoropropylene of the EFEP may be between (31.5 to 54.7):(38 to 64.7):(0.5 to 15), between (31.5 to 54.7):(38 to 64.7):(0.5 to 10), or between (31.5 to 54.7):(38 to 64.7):(13 to 15), with the sum of the values in the molar ratio of ethylene, tetrafluoroethylene, and hexafluoropropylene being 100.

The fluoropolymer is preferably EFEP. The fluoropolymer which is EFEP can provide a composite material having a less colored appearance, and can provide a composite material having a less colored appearance than those containing no zeolite supporting a metal.

The EFEP is a copolymer containing a polymerized unit (Et unit) based on ethylene (Et), a polymerized unit (TFE unit) based on tetrafluoroethylene (TFE), and a polymerized unit (HFP unit) based on hexafluoropropylene (HFP). The EFEP nay b a copolymer consisting only of an Et unit, a TFE unit, and a HFP unit, or may be a copolymer further containing a polymerized unit based on a different fluoromonomer (other than TFE and HFP) or non-fluorinated monomer (other than Et).

A preferred molar ratio of the Et unit, the TFE unit, and he HFP unit is as described above.

The different fluoromonomer and non-fluorinated monomer may be any monomers copolymerizable with Et, TFE, and HFP. C3-C10 fluorovinyl monomers are easy to use, and examples thereof include hexafluoroisobutylene and CH₂═CFC₃F₆H. In an embodiment, fluorovinyl monomers represented by the following formula (I):

CH₂═CH—Rf¹ (1)

(wherein Rf¹ is a C4-C8 perfluoroalkyl group) are preferred. The different non-fluorinated monomer may be a vinyl monomer represented by the following formula (2):

CH₂═CH—R¹ (2)

wherein R¹ may contain any number of carbon atoms, may contain an aromatic ring, and may contain a carbonyl, ester, ether, amide, cyano, hydroxy, or epoxy group; and R¹ is free from a fluorine atom.

The proportion of polymerized units based on the different fluoromonomer and non-fluorinated monomer is preferably 10 mol % or less, more preferable 5 mol % or less, of all the polymerized units constituting the EFEP, while the proportion thereof may be 0.1 mol % or more.

The proportions of the respective monomer units in the above copolymer can be calculated by appropriate combinations of NMR, FT-IR, elemental analysis, and X-ray fluorescence analysis in accordance with the types of the monomers.

The EFEP preferably has a melt flow rate (MFR) of 0.1 to 100 g/10 min, more preferably 0.5 g/10 min or higher, still more preferably 3 g/10 min or higher, while preferably 80 g/10 min or lower, more preferably 45 g/10 min or lower, at 265° C. The MFR is a value determined by the method in conformity with ASTM D7472.

The EFEP has a melting point of preferably 150° C. to 200° C., more, preferably 150° C. to 170° C.

The melting point is a temperature corresponding to the maximum value on a heat-of-fusion curve drawn using a differential scanning calorimeter (DSC) at a temperature-increasing rate of 10° C./min.

The composite material of the present invention, especially in which the fluoropolymer is EFEP, is preferably free from an inorganic component other than the above antimicrobial agent. The composite material can exert sufficient antimicrobial performance even without such an inorganic component. the composite material is particularly preferably free from a flat inorganic compound, especially free from silica, mica, and talc, other than the above antimicrobial agent.

Accordingly, one aspect of the present invention relates to a composite material containing a fluoropolymer and an antimicrobial agent, in which the fluoropolymer and the antimicrobial agent are compounded.

Another aspect of the present invention relates to the composite material described above, in which the fluoropolymer is a fluorinated ethylene propylene copolymer.

A further aspect of the present invention relates to the composite material described above, in which the monomer molar ratio of tetrafluroethylene to hexafluoropropylene is between 70:30 and 99:1, or between 80:20 and 97:3.

A further aspect of the present invention relates to the composite material described above, in which the fluoropolymer is a copolymer of ethylene and tetrafluoroethylene.

A further aspect of the present invention relates to the composite material described above, in which the monomer molar ratio of ethylene to tetrafluoroethylene is between 80:20 and 10:90, between 63:37 and 15:85, or between 62:38 and 20:80.

A further aspect of the present invention relates to the composite material described above, in which the fluoropolymer is a perfluoroalkoxy polymer.

A further aspect of the present invention relates to the composite material described above, in which the fluoropolymer is polyvinylidene fluoride.

A further aspect of the present invention relates to the composite material described above, in which the fluoropolymer is a terpolymer of ethylene, tetrafluoroethylene, and hexafluoropropylene.

A further aspect of the present invention relates to the composite material described above, in which the monomer molar ratio of ethylene, tetrafluoroethylene, and hexafluoropropylene is between (31.5 to 54.7):(38 to 64.7):(0.5 to 15), with the sum of the values in the molar ratio of ethylene, tetrafluoroethylene, and hexafluoropropylene being 100.

A further aspect of the present invention relates to the composite material described above, in which the composite material is free from an inorganic component other than the antimicrobial agent.

A further aspect of the present invention relates to the composite material described above, in which the fluoropolymer is polytetrafluoroethylene in the form of fine powders.

A further aspect of the present, invention relates to the composite material described above, in which the fluoropolymer is polytetrafluoroethylene in the form of molding powders.

A further aspect of the present invention relates to one of the composite materials described above, in which the antimicrobial agent contains elemental ions selected from silver ions, copper ions, and zinc ions.

A further aspect of the present invention relates to one of the composite materials described above, in which the antimicrobial agent contains zeolites.

A further aspect of the present invention relates to one of the composite materials described above, in which the weight ratio of the fluoropolymer to the antimicrobial agent is between 90:10 and 99.99:0.01, between 95:5 and 99.9:0.1, or between 97:3 and 99.8:0.2.

A further aspect of the present invention relates to a method for making one of the composite materials described above, in which the method contains the step of compounding the fluoropolymer and the antimicrobial agent by a co-rotating twin screw extruder.

A further aspect of the present invention relates to a method for making one of the composite materials described above, in which the method contains the step of compounding the fluoropolymer and the antimicrobial agent by a single screw extruder.

A further aspect of the present invention relates to a method for making one of the composite materials described above, in which the method contains the step of compounding the fluoropolymer and the antimicrobial agent by a buss kneader.

A further aspect of the present invention relates to a method for making one of the composite materials described above, in which the method contains the step of compounding the fluoropolymer and the antimicrobial agent by an internal mixer.

A further aspect of the present invention relates to a method for making one of the composite materials described above, in which the method contains the step of compounding the fluoropolymer and the antimicrobial agent by a two roll mill.

A further aspect of the present invention relates to one of the methods described above, in which the fluoropolymer is a fluorinated ethylene propylene copolymer, and the compounding is carried out at a temperature between 580° F. and 700° F.

A further aspect of the present invention relates to one of the methods described above, in which the fluoropolymer is a copolymer of ethylene and tetrafluoroethylene, and the compounding is carried out at a temperature between 580° F. and 650° F.

A further aspect of the present invention relates to one of the methods described above, in which the fluoropolymer is a terpolymer of ethylene, tetrafluoroethylene, and hexafluoropropylene, and the compounding is carried out at a temperature between 390° F. and 550° F.

A further aspect of the present invention relates to one of the methods described above, in which the compounding is carried out at a screw speed between 15 RPM and 1,000 RPM, or between 50 RPM and 100 RPM.

A further aspect of the present invention relates to a tube containing one of the composite materials described above.

A further aspect of the present invention relates to a catheter containing one of the composite materials described above.

### DETAILED DESCRIPTION OF THE INVENTION AND EMBODIMENTS

The detailed description that follows generally describes various exemplary embodiments of the present invention, and should not be considered to be exclusive of other equally effective embodiments, as would be understood by those of ordinary skill in the art. Further, numerous specific details are given in order to provide a thorough understanding of the embodiments and other examples. In some instances, however, well-known methods, procedures, and components have not been described in detail, so as to not obscure the following description. The embodiments and examples disclosed are for exemplary purposes only. Other embodiments and examples may be employed in lieu of, or in combination with, the embodiments and examples disclosed. In what follows, unless otherwise specified, the amounts of the components in a composition are all expressed in weight % relative to the total amount of the composition. Also, where a numerical range is provided, it is understood that all numerical subsets of that range, and all the individual integers contained therein, are provided as part of the invention.

### Experiment 1

Studies were carried out to investigate the antimicrobial effects of the fluoropolymers, specifically FEPs, of the present invention. As the antibacterial agent, Agion® Ak80H from Sciessent was used. Five (5) weight % of this agent was compounded with Neoflon® FEP NP-120, and samples were tested for their antimicrobial performances against E. coli (ATCC #25922) and S. aureus (ATCC #6538) following the Modified ASTM-2180 standard. The sample size of the tested resins was 2 inches×2 inches. The initial inoculum for the tests was at a 10⁶ concentration, which is consistent with what is used for medical testing. (For non-medical testing, the concentration of the initial inoculum would be 10⁵.)

Neoflon® FEP NP-120 is a copolymer of tetrafluoroethylene and hexafluoropropylene, and is widely used in medical-device areas, such as in catheters and other tubes and hoses. It is generally characterized by high extrusion speeds and superior stress crack resistance, and has the typical properties shown in Table 1 below. The monomer ratio of tetrafluoroethylene to hexafluoropropylene in Neoflon® FEP NP-120 is in the range of between 80:20 and 97:3.

Agion® AK80H contains 5% by weight of silver and 13% by weight of zinc in a zeolite carrier.

TABLE 1

| | Measurement | |
|---|---|---|
| Property | Method | Result |
| Melt Flow Rate, g/10 min | ASTM D-2116 | 4.0-10.0 |
| Melting point (DSC), ° C. | ASTM D-2116 | 260-270 |
| Tensile strength, MPa, (minimum) | ASTM D-2116 | 20.0 |
| Elongation, %, (minimum) | ASTM D-2116 | 275 |
| MIT Flex, cycles, avg. | ASTM D-2176 | 30,000 |

To prepare the samples, Neoflon® FEP NP-120 and Agion® AK80H were compounded by a co-rotating twin screw extruder at a temperature up to 630° F. and at a screw speed up to 100 rotations per minute (“RPM”). For tube manufacturing, a single screw extruder may be used, and the temperature may be up to 700° F. and the screw speed up to 60 RPM. The size of a tube depends on the applications intended, and its diameter may be from 1 mm or less, to a few centimeters.

Tables 2 and 3 below show the results obtained from the antimicrobial studies, “Assay (+)” refers to a sample with a positive organism count (that is, contains organisms), while “Assay (−)” refers to a sample without organism. Sample 1, Sample 2, and Sample 3 in Tables 2 and 3 are samples having the same composition but represent different batches.

TABLE 2
Tests against E. coli

| | Organism Count (CFU/ml) | | |
|---|---|---|---|
| | Zero | 24 Hours | |
| | Contact | Contact | Percent |
| Sample Indentification | Time | Time | Reduction** |
| Assay (+) | 3.9 × 10⁶ | 3.6 × 10⁷ | No Reduction |
| Assay (−) | <10* | <10* | N/A |
| FEP NP-120, Natural | | 5.0 × 10⁸ | No Reduction |
| FEP NP-120, 5% AK80H | | <10* | 99.99999% |
| Sample 1 | | | |
| FEP NP-120, 5% AK80H | | <10* | 99.99999% |
| Sample 2 | | | |
| FEP NP-120, 5% AK80H | | <10* | 99.99999% |
| Sample 3 | | | |

Notes:
*<10 = Limits of detection of assay.
**Percent reduction calculated using untreated T24 hour contact time.

TABLE 3
Tests against S. aureus

| | Organism Count (CFU/ml) | | |
|---|---|---|---|
| | Zero | 24 Hours | |
| | Contact | Contact | Percent |
| Sample Indentification | Time | Time | Reduction** |
| Assay (+) | 3.9 × 10⁶ | 4.6 × 10⁶ | No Reduction |
| Assay (−) | <10* | <10* | N/A |
| FEP NP-120, Natural | | 3.2 × 10⁷ | No Reduction |
| FEP NP-120, 5% AK80H | | <10* | 99.9999% |
| Sample 1 | | | |
| FEP NP-120, 5% AK80H | | <10* | 99.9999% |
| Sample 2 | | | |
| FEP NP-120, 5% AK80H | | <10* | 99.9999% |
| Sample 3 | | | |

Notes:
*<10 = Limits of detection of assay.
**Percent reduction calcalated using untreated T24 hour contact time.

For the antimicrobial studies above, Neoflon® FEP NP-130 instead of Neoflon® FEP NP-120 can be used as well, with similar results expected. Neoflon® FEP NP-130 is suitable, among other applications, for extrusion and compression molding that requires an elevated degree of stress crack resistance, and has the typical properties shown in Table 4 below.

TABLE 4

| | Measurement | |
|---|---|---|
| Property | Method | Result |
| Melt Flow Rate, g/10 min | ASTM D-2116 | 2.0-3.6 |
| Melting point (DSC), °C. | ASTM D-2116 | 250-260 |
| Tensile strength, MPa, (minimum) | ASTM D-2116 | 17.2 |
| Elongation, %, (minimum) | ASTM D-2116 | 275 |
| MIT Flex, cycles, (minimum) | ASTM D-2176 | 95,000 |

### Experiment 2

Tube samples were produced by extrusion molding of the respective fluoropolymers and Agion® shown in Table 5, and the antimicrobial performance against S. aureus (ATCC #6538) was determined by the method shown in Table 5. The tube samples tested had a diameter of 3 mm and a length of 150 mm. The results are shown in Table 5 below.

TABLE 5

| | | | | Organism | Organism | | |
|---|---|---|---|---|---|---|---|
| | | Agion | | Count of | Count of | | |
| Fluoropolymer | Agion | Content | | Control | Sample | Bacteria | |
| (Neoflon ®) | Type | (wt %) | Article | (CFU/ml) | (CFU/ml) | Reduction* | ASTM |
| PFA | AK80H | 0.5 | Tubing | 1.37 × 10⁶ | 1.00 × 10 | 99.999% | E2149-13a |
| AP201 | | 1.0 | | 1.37 × 10⁶ | 1.00 × 10 | 99.999% | |
| | | 3.0 | | 1.37 × 10⁶ | 1.00 × 10 | 99.999% | |
| FEP | AK80H | 0.5 | Tubing | 1.37 × 10⁶ | 1.00 × 10 | 99.999% | E2149-13a |
| NP120 | | 1.0 | | 1.37 × 10⁶ | 1.00 × 10 | 99.999% | |
| | | 3.0 | | 1.37 × 10⁶ | 1.00 × 10 | 99.999% | |
| ETFE | AK80H | 0.5 | Tubing | 1.37 × 10⁶ | 1.00 × 10 | 99.999% | E2149-13a |
| EP521 | | 1.0 | | 1.37 × 10⁶ | 1.00 × 10 | 99.999% | |
| | | 3.0 | | 1.37 × 10⁶ | 1.00 × 10 | 99.999% | |
| EFEP | AK80H | 0.5 | Tubing | 1.37 × 10⁶ | 1.00 × 10 | 99.999% | E2149-13a |
| RP5000 | | 1.0 | | 1.37 × 10⁶ | 1.00 × 10 | 99.999% | |
| | | 3.0 | | 1.37 × 10⁶ | 1.00 × 10 | 99.999% | |

Notes:
*Bacteria Reduction = (1-Organism Count of Sample/Organism Count of Control) × 100%
The samples containing EFEP had a less colored appearance than samples free from Agion ®.

In the present invention, the temperature of compounding a fluoropolymer and an antimicrobial agent may be adjusted as appropriate. For example, a composite material containing FEP may be heated at a temperature between 580° F. and 700° F., a composite material containing ETFE may be heated at a temperature between 580° F. and 650° F., and a composite material containing EFEP may be heated at a temperature between 390° F. and 550° F.

The screw speed for compounding may also be adjusted as appropriate in the present invention, depending on such factors as screw designs, temperature settings, and screw extruder sizes. For example, the screw speed may be between 15 RPM and 1,000 RPM, or it may be between 50 RPM and 100 RPM.

In addition to compounding by a co-rotating twin screw extruder or a single screw extruder, the compounding can also be conducted by use of a buss kneader, an internal mixer, a two roll mill, or other apparatuses known in the field.

In an operation of compounding, various materials are mixed, melted and pumped—generally in an extruder or other equipment—and made into forms of pellets. Twin screw extruders, either of a co-rotating design or a counter-rotating design, are the most popular equipment for compounding. The two screws in a twin screw extruder rotate either in the same direction (co-rotating) or in opposite directions (counter-rotating). They can be intermeshing, or non-intermeshing. Single-screw extruders are also used for compounding with improved mixing design. Other types of equipment, such as buss kneaders and mixers, are also found useful in compounding materials, while buss kneaders are applied more in heat- and shear-sensitive areas, such as PVC compounding. An internal mixer and a two roll mill can be used to make such FEP compounds as well. However, they are not continuous technique and are widely used in rubber compounding instead.

Now that exemplary embodiments of the present invention have been shown and described in detail, various modifications and improvements thereon will become readily apparent to those skilled in the art.

It will be understood that one or more of the elements or exemplary embodiments described can be rearranged, separated, or combined without deviating from the scope of the present invention. For ease of description, various elements are, at times, presented separately. This is merely for convenience and is in no way meant to be a limitation.

Further, it will be understood that one or more of the steps described can be rearranged, separated, or combined without deviating from the scope of the present invention. For ease of description, steps are, at times, presented sequentially. This is merely for convenience and is in no way meant to be a limitation.

While the various elements, steps, and exemplary embodiments of the present invention have been outlined above, it is evident that many alternatives, modifications, and variations will be apparent to those skilled in the art. The various elements, steps, and exemplary embodiments of the present invention, as described above, are intended to be illustrative, not limiting. Various changes can be made without departing from the spirit and scope of the present disclosure. Accordingly, the spirit and scope of the present disclosure is to be construed broadly and not limited by the foregoing specification.

No element, act, or instruction used in the description of the present invention should be construed as critical or essential unless explicitly described as such. Also, as used herein, the article “a” is intended to include one or more items. Where only one item is intended, the term “one,” “single,” or similar language is used.

The present invention has industrial applicability in that it provides, among other things, fluoropolymers having antimicrobial capabilities and methods for making them.

1. A composite material, comprising:
a fluoropolymer; and
an antimicrobial agent;
wherein the fluoropolymer and the antimicrobial agent are compounded.

2. The composite material according to claim 1, wherein the fluoropolymer is a fluorinated ethylene propylene copolymer.

3. The composite material according to claim 2, wherein the monomer molar ratio of tetrafluoroethylene to hexafluoropropylene is between 80:20 and 97:3.

4. The composite material according to claim 1, wherein the fluoropolymer is a copolymer of ethylene and tetrafluoroethylene.

5. The composite material according to claim 4, wherein the monomer molar ratio of ethylene to tetrafluoroethylene is between 63:37 and 15:85.

6. The composite material according to claim 1, wherein the fluoropolymer is a perfluoroalkoxy polymer.

7. The composite material according to claim 1, wherein the fluoropolymer is polyvinylidene fluoride.

8. The composite material according to claim 1, wherein the fluoropolymer is a terpolymer of ethylene, tetrafluoroethylene, and hexafluoropropylene.

9. The composite material according to claim 8, wherein the monomer molar ratio of ethylene, tetrafluoroethylene, and hexafluoropropylene is between (31.5 to 54.7):(38 to 64.7):(0.5 to 15), with the sum of the values in the molar ratio of ethylene, tetrafluoroethylene, and hexafluoropropylene being 100.

10. The composite material according to claim 8, wherein the composite material is free from an inorganic component other than the antimicrobial agent.

11. The composite material according to claim 1, wherein the fluoropolymer is polytetrafluoroethylene in the form of fine powders.

12. The composite material according to claim 1, wherein the fluoropolymer is polytetrafluoroethylene in the form of molding powders.

13. The composite material according to claim 1, wherein the antimicrobial agent comprises elemental ions selected from the group consisting of silver ions, copper ions, and zinc ions.

14. The composite material according to claim 1, wherein the antimicrobial agent comprises zeolites.

15. The composite material according to claim 1, wherein the weight ratio of the fluoropolymer to the antimicrobial agent is between 95:5 and 99.9:0.1.

16. A method for making the composite material of claim 1, the method comprising the step of:
compounding the fluoropolymer and the antimicrobial agent by a co-rotating twin screw extruder.

17. A method for making the composite material of claim 1, the method comprising the step of:
compounding the fluoropolymer and the antimicrobial agent by a single screw extruder.

18. A method for making the composite material of claim 1, the method comprising the step of:
compounding the fluoropolymer and the antimicrobial agent by a buss kneader.

19. A method for making the composite material of claim 1, the method comprising the step of:
compounding the fluoropolymer and the antimicrobial agent by an internal mixer.

20. A method for making the composite material of claim 1, the method comprising the step of:
compounding the fluoropolymer and the antimicrobial agent by a two roll mill.

21. The method according to claim 16, wherein:
the fluoropolymer is a fluorinated ethylene propylene copolymer; and
the compounding is carried out at a temperature between 580° F. and 700° F.

22. The method according to claim 16, wherein:
the fluoropolymer is a copolymer of ethylene and tetrafluoroethylene; and
the compounding is carried out at a temperature between 580° F. and 650° F.

23. The method according to claim 16, wherein:
the fluoropolymer is a terpolymer of ethylene, tetrafluoroethylene, and hexafluoropropylene; and
the compounding is carried out at a temperature between 390° F. and 550° F.

24. The method according to claim 16, wherein the compounding is carried out at a screw speed between 15 RPM and 1,000 RPM.

25. The method according to claim 16, wherein the compounding is carried out at a screw speed between 50 RPM and 100 RPM.

26. A tube comprising the composite material according to claim 1.

27. A catheter comprising the composite material according to claim 1.

28. A thin film surface for packaging and medical surface comprising the composite material according to claim 1.

29. An injected molded article used as a medical device comprising the composite material according to claim 1.

30. A blow-molded bottle for medical or food packaging comprising the composite material according to claim 1.
